(19) United States
(12) Patent Application Publication
(10) Pub. No.: US 2026/0232554 A1
(43) Pub. Date: Aug. 13, 2026

(54) ORAL CARE AGENT WITH DIMETHYLGLYCINE

(71) Applicant: Dr. Kurt Wolff GmbH & Co. KG, Bielefeld (DE)

(72) Inventors: Erik SCHULZE ZUR WIESCHE, Bielefeld (DE); Joachim ENAX, Bielefeld (DE); Frederic MEYER, Bielefeld (DE)

(21) Appl. No.: 19/155,886

(22) PCT Filed: Feb. 13, 2024
(86) PCT No.: PCT/EP2024/053527

§ 371 Date: Aug. 12, 2025

(30) Foreign Application Priority Data
Feb. 13, 2023 (DE) 10 2023 103 403.0

(51) Int. Cl. A61K 8/44; A61K 8/24; A61Q 11/00

(52) U.S. Cl. CPC A61K 8/44; A61K 8/24; A61Q 11/00

(57) ABSTRACT

The present invention relates to an oral care composition, in particular an oral care composition for use in the oral cavity, containing dimethylglycine and/or a salt thereof. The invention further relates to the use of such an oral care composition for wound healing and/or preventing and treating gingivitis and/or for regenerating and vitalizing the gums and/or for preventing and treating bleeding gums and/or for caring for the oral mucosa and/or the gums and/or for stimulating salivation. The invention particularly relates to the use of such an oral care composition as a cosmetic.

The present invention relates to an oral care composition, in particular an oral care composition for use in the oral cavity, containing dimethylglycine and/or a salt thereof. The invention further relates to the use of such an oral care composition for wound healing and/or preventing and treating gingivitis and/or for regenerating and vitalizing the gums and/or for preventing and treating bleeding gums and/or for caring for the oral mucosa and/or the gums and/or for stimulating salivation. The invention particularly relates to the use of such an oral care composition as a cosmetic.

The gums (lat. gingiva) are part of the oral mucosa and are characterized, among other things, by the fact that they surround the teeth cervically. Here, the gums surround the tooth neck, thereby sealing the entry point of the tooth into the jawbone of the oral cavity. The gums therefore serve to protect and hold the tooth in place.

The different parts of a natural tooth are the crown, the tooth neck and the tooth root, which are made up of several layers. Of these layers, only the outer enamel (enamelum), which surrounds the dentin and other layers, is normally visible. To bite or grind food, for example, the enamel is very hard. It consists of about 97% by weight hydroxyapatite, which has the following molecular formula: Ca₅(PO₄)₃(OH). Dentin is also considered a dental hard substance and also consists of about two thirds hydroxyapatite. In addition to hydroxyapatite, dentin contains proteins and water and is therefore not as hard as enamel.

Healthy gums are pale pink and show no signs of bleeding. However, if the gums are sensitive to pain, reddish and swollen, this may indicate a disease. Bleeding that occurs after flossing or brushing can also be a sign of changes in the oral mucosa.

In many cases, a lack of oral hygiene leads to bleeding gums. Here, bacteria settle in the oral cavity, especially between the teeth and gums, and can lead to inflammation, which is manifested by bleeding gums.

The most common causes of bleeding gums include gingivitis, periodontitis, necrotizing periodontal diseases such as necrotizing ulcerative gingivitis (NUG) and necrotizing ulcerative periodontitis (NUP), hormonal changes during puberty, pregnancy or menopause, the use of medication for epilepsy, immunosuppressants or antihypertensive drugs. Bleeding gums can also occur as a side effect of diseases such as diabetes mellitus, HIV infection, leukemia or hemophilia, or be caused by fungal or viral infections, as well as due to malnutrition. For example, a vitamin C deficiency can promote bleeding of the gums, as can a protein deficiency. Dehydration can also cause bleeding gums, whereby less saliva is produced, the oral flora becomes unbalanced and consequently tends to become inflamed.

In the case of gingivitis, bacteria conglomerate and multiply in hard-to-reach areas of the oral cavity. Bacterial plaque, also known as plaque, forms on the teeth and an inflammation develops, which is accompanied by bleeding gums and often also bad breath.

If gingivitis remains untreated, the disease can lead to periodontitis, which is also known colloquially as “parodontosis”. Periodontitis is an inflammation of the gums caused by bacteria, which can lead to irreversible destruction of the periodontium. It is also triggered by a bacterial plaque known as plaque. This usually tenaciously adhering bacterial plaque or film can also be referred to as a biofilm. A characteristic of periodontitis is the detectable bone resorption, which is reported to be triggered by enzymes that are supposed to destroy the bacterial biofilm, although these enzymes also lead among other things to the destruction of the patient's own tissue. This can lead to the loss of connective tissue and bone.

Careful oral hygiene and thorough cleaning with conventional oral care products are not always successful in inhibiting the inflammation and swelling underlying bleeding gums and reducing or eliminating bleeding gums. In addition, conventional oral care products cannot improve damage that has occurred, for example, to the periodontium and jawbone as a result of periodontitis.

US 2004/0121025 A1 describes a composition for the systemic treatment of bone resorption comprising a vanadium salt, ipriflavone, at least one homocysteine inhibiting agent and an absorbable calcium salt in a pharmaceutically acceptable carrier. Herein, the homocysteine inhibitor is supposed to block the harmful effects of homocysteine on bone tissue to increase the effectiveness of the components to prevent bone resorption and promote bone formation (i.e., vanadium salt and ipriflavone). The homocysteine inhibitor may be dimethylglycine.

Based on the above, it was an object of the present invention to provide an agent which can reduce the occurrence of bleeding gums, regenerate and vitalize the gums and combat gingivitis, and which is suitable for the (cosmetic) care of the oral mucosa and/or the gums. Furthermore, this composition should be applicable orally in the oral cavity and overcome the disadvantages of the compositions known from the prior art.

This problem was surprisingly solved by the composition according to claim 1 and its use according to claims 8-11. Preferred embodiments are shown in the dependent claims.

According to the invention, the problem is solved by providing an oral care composition, in particular an oral care composition for use in the oral cavity, which contains dimethylglycine and/or a salt thereof.

Accordingly, the present invention relates to an oral care composition, in particular for use in the oral cavity, containing dimethylglycine and/or a salt thereof.

The oral care composition is a cosmetic, pharmaceutical or medicinal preparation, in particular a cosmetic preparation.

An oral care composition according to the present invention is to be understood as a solid, semi-solid or liquid preparation which is intended for application in the oral cavity and/or in the pharynx in order to achieve a local effect there. According to the present invention, an application in the oral cavity and/or in the pharynx is also referred to as oral application.

Accordingly, the oral care composition according to the invention is not intended to be swallowed. No significant amounts and preferably no amount at all of the oral care composition of the invention should enter the gastrointestinal tract.

Solid or semi-solid preparations are preferred, as they remain in the oral cavity for a longer time than liquid preparations.

Solid preparations for use in the oral cavity include tablets, in particular chewable tablets, sublingual, buccal and adhesive tablets, as well as chewable capsules and chewing gums.

Semi-solid preparations for use in the oral cavity include ointments, gels (e.g. oral gels, gels for gingival pockets), creams and pastes (e.g. toothpaste).

Liquid preparations for use in the oral cavity are mouth rinses, gargle solutions, mouthwashes, solutions for use on the gums, solutions and suspensions for use in the oral cavity, drops for use in the oral cavity, sprays for use in the oral cavity and sublingual sprays.

The oral care composition of the present invention preferably acts only (locally) in the oral cavity.

The oral care composition is preferably in the form of a toothpaste, mouth rinse, tablet, chewing gum, mouth gel/oral gel, gel for gingival pockets or mouthwash. The oral care composition is particularly preferably in the form of a toothpaste, mouth rinse or mouth gel/oral gel.

A dentifrice, also referred to as toothpaste, can be used for mechanical tooth cleaning and is a soft or semi-solid composition for oral use, especially on the teeth.

A mouth rinse, also referred to as mouthwash, is a liquid that can be used among other things for the prophylaxis of caries and other diseases of the oral cavity.

A tablet for (local) application in the oral cavity includes chewable tablets, sublingual, buccal and adhesive tablets.

An oral gel is a gel-like composition that can be used to treat complaints/pain, for example in the oral mucosa, gums and lips or to combat dry mouth. The mouth gel is normally applied to the painful area, for example in the case of aphthae on the oral mucosa.

Essentially the same applies to a gel for gingival pockets as to an oral gel, wherein the gel for gingival pockets is essentially applied to the gums and into the pockets formed between the gums and the tooth neck.

In a particularly preferred embodiment, the oral care composition according to the invention is in the form of a toothpaste.

In a further particularly preferred embodiment, the oral care composition according to the invention is in liquid form, in particular as a mouth rinse. In liquid form, the oral care composition according to the invention can be sprayed or gargled. In this case, the liquid oral care composition wets the oral mucosa and thus ensures moisturization.

In a further particularly preferred embodiment, the oral care composition according to the invention is in the form of a mouth gel/oral gel. A mouth gel/oral gel has a viscous consistency, which has the advantage that it can be applied to the mucous membranes like a protective film and also remains thereon. This means that the formulation and active ingredients remain active for a longer period of time. A mouth gel/oral gel is therefore particularly suitable for overnight use.

It has surprisingly been shown that the oral care composition according to the invention containing dimethylglycine and/or a salt of dimethylglycine has excellent efficacy in gum care and in the prevention and healing of gum diseases. In particular, the oral care composition according to the invention reduces bleeding gums, inhibits inflammation and swelling, regenerates and vitalizes the gums and stimulates salivation. In addition, the oral care composition according to the invention containing dimethylglycine and/or a salt of dimethylglycine protects against infections, soothes irritations of the oral mucosa, promotes wound healing and tissue regeneration.

The oral care composition according to the invention is thus excellently suited for wound healing and/or prevention and treatment of gingivitis and/or for regeneration and vitalization of the gums and/or for prevention and treatment of bleeding gums and/or for care of the oral mucosa and/or the gums and/or for stimulation of salivation, also in patients with dry mouth.

Dimethylglycine (N,N-dimethylglycine) is found in plants, animals and humans, although it is only formed in very small quantities in humans. It is formed in a multi-stage biosynthesis of glycine from choline as an intermediate product by transamination of betaine with betaine homocysteine methylase.

N,N-dimethylglycine, also referred to (dimethylamino)acetic acid, is represented by the following chemical formula 1:

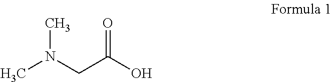

According to the present invention, it was surprisingly found that dimethylglycine and/or a salt of dimethylglycine as a component of an oral care composition for use in the oral cavity is excellently suited for gum regeneration and gum vitalization as well as for gum care and for the prevention and healing of gum diseases, such as gingivitis or periodontitis, and in particular reduces bleeding gums, inhibits inflammation and swelling and contributes to wound healing. Furthermore, it was surprisingly found that dimethylglycine and/or a salt of dimethylglycine can stimulate salivation.

This was particularly surprising because dimethylglycine and/or a salt of dimethylglycine had not previously been described in the field of dental care/gum care/mouth care.

According to the invention, not only dimethylglycine is used, but also its salts, solvates and hydrates. These are preferably pharmaceutically or cosmetically acceptable salts of dimethylglycine. The salt is particularly preferably a water-soluble salt with a solubility in water of at least 10 g/l at 20° C.

In a preferred embodiment, the salt of dimethylglycine is an alkali metal, alkaline earth metal or ammonium salt of dimethylglycine.

Examples are sodium, potassium, calcium, magnesium and ammonium salts. In the case of ammonium salts, the ammonium cation carries one to four alkyl groups, each having independently of one another 1 to 4 carbon atoms. Preferred are the sodium and potassium salt of dimethylglycine, in particular the sodium salt of dimethylglycine, namely sodium dimethylglycinate (sodium N,N-dimethylglycinate).

In an alternatively preferred embodiment, the salt of dimethylglycine may be the salt of an inorganic and/or organic acid with dimethylglycine.

Examples of salts of dimethylglycine with an inorganic acid are the hydrochloride, hydrobromide, hydroiodide, hydrogen sulfate, sulfate, hydrogen sulfite, sulfite, hydrogen carbo-nate, carbonate, monophosphate, diphosphate and triphosphate of dimethylglycine as well as mixtures thereof. Particularly preferred is the hydrochloride of dimethylglycine, namely dimethylglycine hydrochloride (N,N-dimethylglycine hydrochloride).

Examples of salts of dimethylglycine with an organic acid are the acetate, lactate, citrate, succinate, fumarate, maleate and benzoate of dimethylglycine as well as mixtures thereof.

According to the invention, the dimethylglycine and/or salt of dimethylglycine is preferably selected from the group consisting of dimethylglycine, sodium dimethylglycinate and dimethylglycine hydrochloride.

According to the invention, it is believed that dimethylglycine and/or a salt of dimethylglycine leads to gum regeneration.

The composition according to the invention contains dimethylglycine and/or a salt of dimethylglycine preferably in an amount of from 0.00001% by weight to 10.0% by weight, based on the total weight of the oral care composition. In a preferred embodiment, the composition according to the invention contains dimethylglycine and/or a salt of dimethylglycine in an amount of from 0.00001% by weight to 5% by weight, preferably from 0.0001% by weight to 5% by weight, preferably from 0.0001% by weight to 2.0% by weight, more preferably from 0.0005 to 1% by weight, in each case based on the total weight of the oral care composition.

In a preferred embodiment of the invention, the oral care composition according to the invention may contain dimethylglycine and/or a salt of dimethylglycine in an amount of from 0.00001% by weight, 0.0001% by weight, 0.0002% by weight, 0.0005% by weight, 0.001% by weight, 0.002% by weight or 0.005% by weight to 10% by weight, 5% by weight, 2% by weight, 1% by weight, 0.5% by weight, 0.2% by weight, 0.1% by weight, 0.05% by weight, 0.02% by weight or 0.01% by weight, in each case based on the total weight of the oral care composition.

In a further preferred embodiment of the invention, the oral care composition according to the invention may contain dimethylglycine and/or a salt of dimethylglycine in an amount of about 0.005% by weight or about 0.01% by weight or about 0.1% by weight or about 0.5% by weight or about 1.0% by weight or about 2.0% by weight or about 2.5% by weight or about 3.5% by weight, in each case based on the total weight of the oral care composition.

In a further preferred embodiment of the invention, the oral care composition according to the invention is a toothpaste containing dimethylglycine and/or a salt of dimethylglycine in an amount of about 0.005% by weight or about 0.1% by weight or about 1.0% by weight or about 2.0% by weight, in each case based on the total weight of the oral care composition.

In a further preferred embodiment of the invention, the oral care composition according to the invention is a mouth rinse containing dimethylglycine and/or a salt of dimethylglycine in an amount of about 0.01% by weight or about 0.5% by weight or about 2.5% by weight or about 3.5% by weight, in each case based on the total weight of the oral care composition.

Preferably, the oral care composition according to the invention contains dimethylglycine and/or a salt of dimethylglycine as a chemically pure substance, including the respective solvates and hydrates (e.g. the dihydrate of sodium dimethylglycinate), since the purity of the composition can thus be increased and the occurrence of undesirable side effects can be reduced. For this reason, the oral care composition according to the invention preferably contains the chemical derivatives of dimethylglycine selected from methylglycine, trimethylglycine, (2-hydroxyethyl)-trimethylammonium, and trimethyl-hydroxybutyrobetaine, in concentrations of less than 0.01% by weight, based on the total weight of the composition. Particularly preferably, the compositions according to the invention are completely free of these derivatives.

In a preferred embodiment, the oral care composition according to the invention does not contain any N-alkyl derivatives of dimethylglycine.

In a preferred embodiment, the oral care composition according to the invention further comprises one or more calcium phosphate compound(s).

The calcium phosphate compound is preferably selected from the group consisting of monocalcium phosphate monohydrate (MCPM), monocalcium phosphate anhydrate (MCPA), dicalcium phosphate dihydrate (DCPD, brushite), dicalcium phosphate anhydrate (DCPA, monetite), octacalcium phosphate (OCP), α-tricalcium phosphate (α-TCP), β-tricalcium phosphate (β-TCP), amorphous calcium phosphate (ACP; also referred to as CPP-ACP complex=casein phosphopeptide—amorphous calcium phosphate), calcium-deficient hydroxyapatite (CDHA), hydroxyapatite (HA or HAP), tetracalcium phosphate (TTCP) and calcium pyrophosphate. Particularly preferably the calcium phosphate compound is hydroxyapatite.

The calcium phosphate compound, in particular hydroxyapatite, remineralizes the enamel and therefore acts in the prophylaxis of caries. In addition, the calcium phosphate compound, in particular hydroxyapatite, protects against pain-sensitive teeth.

Hydroxyapatite has the chemical formula Ca₅(PO₄)₃(OH) and is also known as hydroxyapatite. It is a mineral from the mineral class of phosphates, which crystallizes in a hexagonal crystal system.

Hydroxyapatite is also a member of the apatite group and forms a complete series of mixed crystals with chlorapatite and fluorapatite.

The hydroxyapatite contained in the present oral care composition is preferably produced synthetically. This means that the hydroxyapatite used is preferably not obtained by burning out the organic components from animal material such as bone.

In a preferred embodiment, hydroxyapatite is present in pure form. According to the invention, a pure form is present precisely when the ions (Ca²⁺, PO₄³⁻ and OH⁻) contained in the hydroxyapatite are each substituted by less than 1%, preferably less than 0.5%, more preferably less than 0.1% by one or more other ions. For example, in pure hydroxyapatite the Ca²⁺ ions are substituted by, for example, Mg²⁺ or Zn²⁺ and the OH⁻ ions are substituted by, for example, fluoride or chloride by less than 1%, preferably less than 0.5%, more preferably less than 0.1%.

Thus, the composition according to the invention comprises (pure) hydroxyapatite (Ca₅(PO₄)₃(OH)) as the only apatite component.

It has been shown that by using hydroxyapatite as an apatite component, the addition of fluorides such as CaF₂ and/or fluorapatite, which were previously used to harden enamel and combat caries, for example, can be dispensed with in the present oral care composition. This means that the negative effects that are currently being discussed in connection with fluoride-containing dental care and oral products can be avoided in any case. One of these negative effects is fluorosis, which is caused by excessive fluoride intake and triggers symptoms such as nausea, vomiting and diarrhea. Other examples are bone fluorosis, which is characterized by a thickening of the outer bone layer and the associated loss of elasticity and resilience of the bones, and enamel fluorosis, which can be recognized by the appearance of whitish enamel spots on the tooth surface. It is also reported that swallowing high doses of dental care products can cause acute fluoride poisoning, particularly in children, which can sometimes even be fatal. It is also reported that the WHO is not in a position to set a value for a daily fluoride requirement, as fluoride is not an essential trace element and there are therefore no diagnostic parameters and no proof of the existence of clinical symptoms of a “fluoride deficiency”.

Furthermore, the oral care composition according to the invention comprises the calcium phosphate compound, in particular hydroxyapatite, preferably in a particulate form, i.e. the calcium phosphate compound, in particular the hydroxyapatite, is preferably present in a particulate or particle form. Preferably, it is a micro- and/or nanoparticulate calcium phosphate compound, preferably micro- and/or nanoparticulate hydroxyapatite.

The X₅₀ value of the volume-based particle size distribution Q₃(x) of the hydroxyapatite is 1 to 100 nm in the case of nanoparticulate hydroxyapatite and more than 100 nm in the case of microparticulate hydroxyapatite, the X₅₀ value of the volume-based particle size distribution being measured by means of laser diffraction. Preferably, the X₅₀ value of the volume-based particle size distribution Q₃(x) of the hydroxyapatite is 1.0 nm to 100.0 μm, preferably 10 nm to 10 μm, more preferably 50 nm to 5 μm, particularly preferably 100 nm to 5500 nm. Particularly preferred is microparticulate hydroxyapatite with a volume-based particle size distribution of 1.0 to 15.0 μm, in particular 1.2 to 12.0 μm or 1.5 to 10.0 μm, and most preferably 2.0 to 5.0 μm, the X₅₀ value of the volume-based particle size distribution being measured by laser diffraction.

For this purpose, a sample of hydroxyapatite is first sonicated in an ultrasonic homogenizer with an energy performance of 96 W for 9 minutes and then for another 3 minutes in a device for sample preparation. The subsequent particle size distribution measurement (laser diffraction) is carried out in a particle size determination instrument at a temperature of 25° C.±0.3° C. and the corresponding values are calculated according to the Mie theory. The measuring instruments used are exclusively commercially available devices.

In a preferred embodiment according to the invention, the hydroxyapatite has a hexagonal crystal lattice in which the length of the a-axis is 0.930 to 0.950 nm, preferably 0.933 to 0.948 nm, particularly preferably 0.936 to 0.945 nm and the length of the c-axis is 0.680 to 0.700 nm, preferably 0.682 to 0.696 nm, particularly preferably 0.685 to 0.692 nm. The lengths of the a-axis and the c-axis are determined by a Rietveld evaluation of the corresponding X-ray powder diffractograms. The X-ray powder diffractograms themselves are obtained by means of a measurement with a conventional powder diffractometer at the routine settings.

It is further preferred that the hydroxyapatite used according to the invention has a largely spherical crystal morphology. In particular, it is preferred that the hydroxyapatite does not have a needle-shaped crystal morphology. Such a needle-shaped crystal morphology could have undesirable disadvantages, similar to asbestos.

In a preferred embodiment, the hydroxyapatite is present in aggregated form. In this case, aggregation is understood to mean an agglomeration of molecules or particles to form a larger structure, the aggregate. This agglomeration or aggregate is caused and held together by various forces and/or types of bonds, such as ionic bonding, van der Waals forces, intermolecular forces or other types of chemical bonds. The degree of aggregation and also the size of the aggregate can be determined using scanning electron microscopy. It is preferred that no nanoparticles can be detected in the aggregated form of hydroxyapatite even after high energy input. Particles with a size of less than 100 nm are referred to as nanoparticles. With regard to nanoparticles, it is not yet known whether they can have an adverse effect on humans, for example after potential penetration of the intestinal wall.

A hydroxyapatite suitable according to the invention is described, for example, in application DE 10 2016 114 189.5.

A hydroxyapatite suitable according to the invention is available, for example, under the name KALI-DENT 100 1st B (PF210715G1) or KALIDENT POWDER 100-B (PF210715G4) from Kalichem Italia s.r.l., Rezzato, Italy.

Preferably, the oral care composition according to the invention contains one or more calcium phosphate compound(s), preferably hydroxyapatite, in an amount of 0.01 to 40% by weight, preferably 0.1 to 30% by weight, more preferably 0.5 to 20.0% by weight, based on the total weight of the oral care composition according to the invention.

In a preferred embodiment of the invention, the oral care composition according to the invention may contain one or more calcium phosphate compound(s), preferably hydroxyapatite, in an amount of from 0.01% by weight, 0.02% by weight, 0.05% by weight, 0.1% by weight, 0.2% by weight, 0.5% by weight, 1.0% by weight, 1.5% by weight, 2.0% by weight, 5.0% by weight, 10.0% by weight, 15.0% by weight, 20.0% by weight, 25.0% by weight, 30.0% by weight, 35.0% by weight or 40% by weight, in each case based on the total weight of the oral care composition.

It is known that the amount of calcium phosphate compound(s), preferably hydroxyapatite, can also depend on the form in which the oral care product (toothpaste, mouth gel, etc.) is present. For example, a toothpaste may contain a larger amount of calcium phosphate compound(s), preferably hydroxyapatite, than a mouth rinse.

In a preferred embodiment of the invention, the oral care composition according to the invention is a toothpaste containing dimethylglycine and/or a salt of dimethylglycine in an amount of about 0.005% by weight, based on the total weight of the oral care composition. Preferably, this oral care composition does not contain any calcium phosphate compound, preferably no hydroxyapatite.

In a preferred embodiment of the invention, the oral care composition according to the invention is a toothpaste containing dimethylglycine and/or a salt of dimethylglycine in an amount of about 0.1% by weight and a calcium phosphate compound, preferably hydroxyapatite, in an amount of 20% by weight, in each case based on the total weight of the oral care composition.

In a preferred embodiment of the invention, the oral care composition according to the invention is a toothpaste containing dimethylglycine and/or a salt of dimethylglycine in an amount of about 1.0% by weight and a calcium phosphate compound, preferably hydroxyapatite, in an amount of 10% by weight, in each case based on the total weight of the oral care composition.

In a preferred embodiment of the invention, the oral care composition according to the invention is a toothpaste containing dimethylglycine and/or a salt of dimethylglycine in an amount of about 2.0% by weight and a calcium phosphate compound, preferably hydroxyapatite, in an amount of 1.0% by weight, in each case based on the total weight of the oral care composition.

In a further preferred embodiment of the invention, the oral care composition according to the invention is a mouth rinse containing dimethylglycine and/or a salt of dimethylglycine in an amount of about 0.01% by weight and a calcium phosphate compound, preferably hydroxyapatite, in an amount of 0.1% by weight, in each case based on the total weight of the oral care composition.

In a further preferred embodiment of the invention, the oral care composition according to the invention is a mouth rinse containing dimethylglycine and/or a salt of dimethylglycine in an amount of about 0.5% by weight and a calcium phosphate compound, preferably hydroxyapatite, in an amount of 5.0% by weight, in each case based on the total weight of the oral care composition.

In a preferred embodiment of the invention, the oral care composition according to the invention is a toothpaste containing dimethylglycine and/or a salt of dimethylglycine in an amount of about 2.5% by weight, based on the total weight of the oral care composition. Preferably, this oral care composition does not contain any calcium phosphate compound, preferably no hydroxyapatite.

In a preferred embodiment of the invention, the oral care composition according to the invention is a toothpaste containing dimethylglycine and/or a salt of dimethylglycine in an amount of about 3.5% by weight, based on the total weight of the oral care composition. Preferably, this oral care composition does not contain any calcium phosphate compound, preferably no hydroxyapatite.

In a preferred embodiment, the oral care composition according to the invention may further comprise one or more surfactant(s).

Surfactants are amphipathic molecules and consist of an oil-soluble and a water-soluble part. In other words, surfactants have a lipophilic and a hydrophilic part. Surfactants are classified according to the hydrophilic part and its charge. There are the classes of non-ionic, cationic, anionic and amphoteric surfactants.

The hydrophilic part of a non-ionic surfactant is not charged. A non-ionic surfactant does not contain a dissociable group, but one or more polar groups such as ethers, ketones and alcohols. Frequently used non-ionic surfactants are, for example, polyalkylene glycol ethers.

Cationic surfactants are positively charged in their hydrophilic part. Most cationic surfactants are quaternary ammonium compounds with halides as counterions, such as distearyldimethylammonium chloride.

The hydrophilic part of an anionic surfactant is negatively charged. The anionic surfactants often have carboxy, alkoxy, sulphonate or sulphate groups with alkali metal or alkaline earth metal ions as counterions. An example of an anionic surfactant is sodium laurylsulfate.

Depending on the pH, the hydrophilic part of an amphoteric surfactant comprises at least one group that is or can become positively charged and at least one group that is or can become negatively charged. The group that is or can become positively charged is, for example, an amine or ammonium group. The group that is or can become negatively charged is, for example, a carboxy, alkoxy, sulphonate or sulphate group. Examples of amphoteric surfactants are amphoacetates, amphodiacetates, amphopropionates, ampho-dipropionates, sulfobetaines and hydroxysultaines.

In a preferred embodiment, the surfactant contained in the oral care composition according to the invention is an anionic or an amphoteric surfactant.

Preferably, the surfactant is selected from sodium lauryl sulfate, sodium cocoamphopropionate, cocamidopropyl betaine, sodium methyl cocoyl taurate, sodium cocoyl glycinate, sodium myristoyl sarcosinate, cocamidopropyl hydroxy sultaine and sodium cocoamphoacetate or mixtures thereof

In a preferred embodiment, the oral care composition according to the invention does not contain sodium lauryl sulfate.

Preferably, the surfactant is selected from the group consisting of betaines, glycinates, sarcosinates, sulfates and taurates.

Preferably, the oral care composition according to the invention contains one or more surfactant(s) in an amount of 0.05 to 5% by weight, more preferably 0.07 to 4% by weight, in particular 0.1 to 3% by weight, based on the total weight of the oral care composition.

In a preferred embodiment, the oral care composition according to the invention may contain one or more pharmaceutical or cosmetic excipients. These pharmaceutical or cosmetic excipients are described, for example, in Toothpastes, Monographs in Oral Science, Vol. 23, 1st edition, 2013.

According to the invention, the pharmaceutical or cosmetic excipients are preferably antimicrobial (in particular antibacterial) substances, pH regulators, cleaning agents (also known as abrasives), flavouring agents, gelling agents, emulsifiers or caring agents.

Antimicrobial substances are substances that can kill microorganisms such as bacteria or greatly reduce their proliferation. In addition to antimicrobial substances with an unspecific defense against bacteria and fungi, there are also substances that only work against, for example, specific bacteria. The use of antimicrobial substances can also combat, for example, bad breath. Preferably, antimicrobial substances can be contained in an amount of 0.01 to 1.0% by weight, preferably 0.05 to 0.5% by weight in the oral care composition according to the invention. Examples of the antimicrobial, in particular antibacterial substances used in oral care are zinc compounds such as zinc chloride, zinc citrate and zinc PCA as well as chlorhexidine, triclosan, cetylpyridinium chloride and stannous chloride.

Preferred antimicrobial, in particular antibacterial, substances according to the invention comprise zinc compounds such as zinc chloride, zinc citrate and zinc PCA, lactoferrin and lysozyme.

In a preferred embodiment, the oral care composition according to the invention does not contain chlorhexidine. In a further preferred embodiment, the oral care composition according to the invention does not contain any quaternary ammonium compounds, in particular no cetylpyridinium chloride. pH regulators are substances that can adjust a certain pH range, preferably a neutral range of pH 6.5 to 7.5. If the composition is too acidic, there would be a risk of demineralization of the tooth structure (erosion). Examples of pH regulators are sodium hydroxide (NaOH) or phosphoric acid (H₃PO₄), which can be used according to the desired pH. Sodium hydroxide can be added to raise a pH that is too low, while phosphoric acid can be added if the pH is too high.

Alternatively, a mixture of sodium phosphate and disodium phosphate can be used. A particular advantage of this buffer system is the use of phosphate, which is also a component of the hydroxyapatite in the enamel and can be stored there, thereby stabilizing it. In addition, the solubility equilibrium is shifted towards remineralization. Preferably, the mixture of sodium phosphate and disodium phosphate is present in a ratio of 1:1. Therefore, in a preferred embodiment, the preparation according to the invention comprises a mixture of sodium phosphate and disodium phosphate, preferably in a ratio of 1:1.

Cleaning agents, also known as abrasives, cleaning or grinding agents, usually remove plaque and harmful bacteria from the tooth surface together with the toothbrush during the tooth cleaning process and can also ensure whitening. Cleaning agents can be contained in the oral care composition according to the invention, preferably in an amount of up to 10% by weight, based on the total weight of the oral care composition. Examples of cleaning agents are precipitated chalk, marble powder and/or silicate compounds such as silica. In a preferred embodiment, the oral care composition according to the invention contains no silicate compound, in particular no silica.

Flavouring agents can give the oral care composition according to the invention the desired taste. In addition, flavouring agents can be saliva-stimulating, whereby the moisture of the saliva can have a positive influence on the remineralization of the tooth. An example of a saliva-stimulating flavouring agent is pellitorin, in particular trans-pellitorin. Other examples of flavouring agents include mint and/or citrus flavouring, preferably mint and citrus flavouring, more preferably mint and citrus flavouring in a ratio of 1:1.

Gel forming agents, also termed gelling agents, are responsible for the formation of a stable gel and give the oral care composition, such as a toothpaste, a pleasant texture and sufficient flowability so that it can be conveniently applied to or spread on the mucous membrane or the gums. In general, all gelling agents known in the pharmaceutical and/or cosmetic field for the preparation of stable gel formulations can be used in the context of the invention. Preferably, hydrophilic gelling agents are used. Examples of suitable gelling agents are natural gelling agents such as pectin, agarose, gelatine and casein or modified natural gelling agents such as cellulose derivatives including methylcellulose, hydroxymethylcellulose, hydroxyethylcellulose (HEC), hydroxymethylpropylcellulose (HPMC) and carboxymethylcellulose, or fully synthetic gelling agents such as polyvinyl alcohols, poly(meth)acrylic acids, polyacrylamide, polyvinylpyrrolidone, polypropylene glycol, polyethylene glycol and poloxamers. Cellulose derivatives and poloxamers are preferred because of the higher stability and adequate viscosity of the gels obtained. Poloxamers are most preferred for reasons of gel stability and because of their function as solubilizers. Suitable poloxamers are poloxamer 407 (e.g. trade name Pluronic F 127; Kolliphor P 407), or poloxamer 188 (e.g. trade name Pluronic F 68; Kolliphor P 188).

Depending on the amount used, gel forming agents or gelling agents can generally also act as thickening agents. In particular, cellulose derivatives are also used as thickeners in toothpastes and mouth rinses.

Suitable emulsifiers which may be present in the oral care composition according to the invention correspond to the surfactants mentioned herein and comprise, for example, PEG-40 Hydro-genated Castor Oil.

Suitable conditioning agents that may be present in the oral care composition according to the invention include allantoin, bisabolol, hyaluron and panthenol.

In a further preferred embodiment, the oral care composition according to the invention further comprises caffeine.

Furthermore, dental care compositions often contain fluoride compounds such as sodium fluoride, amine fluorides or zinc fluoride. In a particularly preferred embodiment, the oral care composition according to the invention does not contain any fluoride compound and is thus fluoride-free.

In a further preferred embodiment, the oral care composition according to the invention does not contain titanium dioxide.

In a particularly preferred embodiment, the oral care composition according to the invention contains no fluoride compound, no chlorhexidine, no titanium dioxide, no sodium lauryl sulphate, no silicate compound, in particular no silica, and/or no quaternary ammonium compound, in particular no cetylpyridinium chloride.

In a preferred embodiment, the oral care composition further comprises antibacterial substances and/or cleaning agents.

Exemplary and preferred pharmaceutical or cosmetic excipients according to the invention comprise 1,2-hexanediol, 1,2-octanediol, allantoin, bisabolol, purified water, glycerol, houseleek extract (Sempervivum tectorum extract), hyaluron or hyaluronic acid, hydroxyacetophenone, hydroxyethylcellulose, lactoferrin, lysozyme, panthenol, PEG-40 hydrogenated castor oil, propylene glycol, sorbitol, stevia extract (Stevia rebaudiana extract), xylitol and zinc salts or zinc compounds.

1,2-hexanediol and/or 1,2-octanediol primarily have a moisturizing and conditioning effect, but also have a preservative effect. For example, a mixture of 1,2-hexanediol and 1,2-octanediol is commercially available under the trade name SymDiol 68® from the company Symrise.

According to the invention, allantoin has a soothing, regenerative and healing effect on the tissue. Allantoin can thus prevent cracks in the tongue, lips and palate.

Bisabolol has an anti-inflammatory effect on the skin and mucous membranes and is used for skin regeneration and wound healing.

According to the invention, glycerol (propanetriol) serves both as a solvent and as a moisturizing agent. In a preferred embodiment, the oral care composition according to the invention for use in the oral cavity comprises glycerol 86.5%.

The houseleek extract is also known as Sempervivum tectorum extract. The houseleek extract also has a soothing, regenerative and healing effect on the tissue and thus serves as a care agent for the oral mucosa. Houseleek extract is, for example, commercially available under the trade name Houseleek Bio Extract ‘C’ G (CH) P-00025259 from the company Botanica GmbH.

Hyaluronic acid (HA) or a salt thereof is used according to the invention for moisturizing, for gum care and for the prevention and healing of gum diseases. Hyaluronic acid is a natural protein that occurs as a component in the connective tissue of the human organism. Hyaluronic acid or a salt thereof protects against infections, relieves irritations of the oral mucosa, promotes wound healing and tissue regeneration. In addition, hyaluronic acid or a salt thereof reduces bleeding gums, inhibits inflammation and swelling.

In a preferred embodiment, the preparation according to the invention for use in the oral cavity comprises the sodium salt of hyaluronic acid, which is also known as sodium hyaluronate.

Hydroxyacetophenone has an anti-oxidative effect. Hydroxyacetophenone also has preservative properties. Hydroxyacetophenone is commercially available under the trade name SymSave H®, for example.

Hydroxyethyl cellulose serves as a viscosity regulator and thus produces the preferred viscous or gel-like consistency of the preparation for application in the oral cavity. Alternatively, carboxymethyl cellulose, alginates, xanthan gum and/or carrageenan can be used as viscosity regulators.

Lactoferrin has both antiviral and antimicrobial properties and is effective in the treatment of chronic periodontitis.

Lysozyme, also known as muramidase, is an enzyme that occurs in animals and humans as part of the innate immune system and has an antibacterial effect.

Panthenol, also known as dexpanthenol, pantothenol, D-panthenol or provitamin B5, is converted to pantothenic acid (vitamin B5) in the body. Pantothenic acid is a component of coenzyme A and therefore plays an important role in dermatological metabolism. Panthenol has an important function in the wound healing of the skin. Panthenol also has an antipruritic and anti-inflammatory effect.

PEG-40 Hydrogenated Castor Oil is an emulsifier.

Propylene glycol is a humectant and thus serves to moisturize the oral mucosa.

Xylitol, sorbitol and the stevia extract (Stevia rebaudiana extract) are sweeteners and serve to improve the taste of the preparation according to the invention. The stevia extract is known as E 960, sorbitol as E 420 and xylitol as E 967 in accordance with Regulation (EU) No. 231/2012.

Xylitol also serves as a humectant. Xylitol also stimulates saliva formation and promotes the neutralization of microbial acids and the remineralization of teeth. Xylitol can minimize the number of caries bacteria and inhibit their growth. It therefore also has a supportive effect in the prophylaxis of caries. The oral care composition according to the invention may contain xylitol in an amount of 0.1 to 10% by weight, preferably 0.7 to 8% by weight, based on the total weight of the oral care composition. In addition to xylitol, the oral care composition according to the invention may contain other sugar alcohols such as sorbitol.

Zinc salts/zinc compounds act as antimicrobial, in particular antibacterial substances and preferably comprise zinc chloride, zinc citrate or zinc PCA, i.e. the combination of zinc with pyrrolidone carboxylic acid (PCA). They reduce plaque and prevent tartar formation and bad breath.

In a particularly preferred embodiment, the composition for use in the oral cavity according to the invention has a pH of 5.0-7.5 and comprises the following ingredients:

| | | |
|---|---|---|
| Glycerol | 8.0-12.0% | by weight |
| Sorbitol | 3.0-5.0% | by weight |
| Hydroxyapatite | 18.0-22.0% | by weight |
| Dimethylglycine | 0.0005-0.005% | by weight |
| Cleansing silica | 3.0-5.0% | by weight |
| Cellulose gum | 6.0-8.0% | by weight % |
| Carboxymethylcellulose | 0.05-0.15% | by weight |
| Sodium methylcocoyl taurate | 0.8-1.2% | by weight. % |
| Flavour | 0.4-0.6% | by weight |
| Phenoxyethanol | 0.2-0.4% | by weight |
| Potassium sorbate | 0.15-0.25% | by weight |
| Sodium myristoyl sarcosinate | 0.8-1.2% | by weight |
| Sodium saccharin | 0.4-0.6% | by weight |
| Zinc chloride | 0.05-0.15% | by weight |
| Carrageenan | 0.05-0.15% | by weight |
| Remainder: demineralized water | | |

The oral care composition according to the invention can be prepared by known processes.

The present invention further relates to the use of the oral care composition described herein. The use is in particular a cosmetic or medical use, preferably a cosmetic use.

The oral care composition according to the invention is thus preferably used as a cosmetic. In one aspect, the present invention relates to the use, in particular the cosmetic use, of the oral care composition described herein for the care of oral and pharyngeal soft tissues, in particular for the care of gums, gingival pockets, tongue, cheek, pharynx and palate. The term “care” in this context comprises the cleaning of gums, gingival pockets, tongue, cheek, pharynx and palate as well as the application (with/without rinsing) to gums, gingival pockets, tongue, cheek, pharynx and palate.

In a further aspect, the present invention relates to the use of the oral care composition described herein for the prevention and/or therapy of oral diseases selected from gingivitis, periodontitis, mucositis, peri-implantitis, peri-implant mucositis, aphthae, fungal thrush, oral fistulae, receding gums, bad breath, ulcers and saliva deficiency.

Encompassed by the present invention is thus an oral care composition as described herein for use in the prevention and/or therapy of oral diseases selected from gingivitis, periodontitis, mucositis, peri-implantitis, peri-implant mucositis, aphthae, fungal thrush, oral fistulae, receding gums, bad breath, ulcers and saliva deficiency.

In still another aspect, the present invention relates to the use, in particular the cosmetic use, of the oral care composition described herein for improving the oral soft tissues, in particular the oral mucosa and/or the gums. The term “improvement of the oral soft tissues” in this context refers, inter alia, in relation to the gums (soft tissues), to gum regeneration, gum vitalization, reduction of bleeding gums, reduction and healing of gingivitis.

Finally, the present invention also relates to the use of the oral care composition described herein for the prevention and/or therapy of bacterial and/or viral infection and/or mycoses.

Encompassed by the present invention is thus an oral care composition as described herein for use in the prevention and/or therapy of a bacterial and/or viral infection and/or mycoses.

In one embodiment of the uses, the oral care composition according to the invention remains in the oral cavity after application (leave-on product).

In a further embodiment of the uses, the preparation according to the invention is used as often as necessary.

The following examples are intended to illustrate the invention without, however, restricting it to the specific formulations.

### BRIEF DESCRIPTION OF THE FIGURES

FIG. 1

FIG. 1 shows the cell viability after 24 h, 48 h and 72 h of cultivation and a clear increase after 48 h and 72 h due to the addition of DMG is observed. Cell viability was determined using the MTT assay. The absorption was measured in quadruplicate and normalized to the control at 24 h. The mean value with standard deviation is indicated.

FIG. 2

FIG. 2 shows the cell proliferation after 24 h, 48 h and 72 h of cultivation and a clear increase after 48 h and 72 h due to the addition of DMG is observed. Cell proliferation was determined using the CyQUANT assay. The fluorescence was measured in triplicate and normalized to the control at 24 h. The mean value with standard deviation is indicated.

FIG. 3

FIG. 3 shows the migration over time and using the different DMG concentrations. Migration or wound closure is each significantly faster with DMG compared to the control without DMG. The wound was created at 0 h and images were taken after 16 h, 20 h and 24 h of cultivation. Based on the images, the cultivation surface that was not yet colonized with cells was determined using the ImageJ software. The measured values are normalized to the time point 0 h (maximum size of the wound) and given in %.

FIG. 4

FIG. 4 shows the gene expression of VEGF after 24 h of cultivation and a significant increase in gene expression due to the addition of DMG is observed. The relative gene expression of VEGF determined using qRT-PCR (triplicate determination, normalized to the respective gene expression of a constitutively expressed gene; GAPDH—glyceraldehyde-3-phosphate dehydrogenase) is shown. The mean value with standard deviation is indicated.

FIG. 5

FIG. 5 shows the gene expression of IL-6 after 24 h of cultivation. The relative gene expression determined using qRT-PCR (triplicate determination, normalized to the respective gene expression of a constitutively expressed gene; GAPDH—glyceraldehyde-3

### EXPERIMENTAL PART

The following compositions were prepared by means known to a person skilled in the art. The quantity of the components was selected in each case so that their weight proportion in the premix corresponds to the indicated weight proportions.

### Example 1

Examples for the composition of the oral care compositions according to the invention in the form of toothpastes (in % by weight):

| | Example 1 | Example 2 | Example 3 | Example 4 |
|---|---|---|---|---|
| Glycerol | 10.0 | 5.0 | 10.0 | 14.0 |
| Sorbitol | 4.0 | 3.0 | 10.0 | 6.0 |
| Hydroxyapatite | 20.0 | 10.0 | 1.0 | — |
| Dimethylglycine | 0.1 | 1.0 | 2.0 | 0.005 |
| Xylitol | — | 2.0 | 8.0 | 10.0 |
| Cleansing silica | 4.0 | 3.0 | 2.0 | 8.0 |
| Cellulose gum | 7.0 | 1.0 | 1.0 | 3.0 |
| Hydroxyethyl | — | 0.2 | 1.0 | 0.5 |
| cellulose | | | | |
| Carboxymethyl | 0.1 | 0.2 | 1.0 | 0.5 |
| cellulose | | | | |
| Sodium methylcocoyl | 1.0 | 2.0 | 0.5 | 2.0 |
| taurate | | | | |
| Sodium sulfate | — | — | 0.5 | — |
| 1,2-hexanediol | — | — | 0.2 | — |
| Caprylyl glycol | — | — | 0.5 | — |
| Flavour | 0.5 | | 0.3 | 1.5 |
| Sodium cocoyl | — | — | 0.2 | — |
| glycinate | | | | |
| Sodium chloride | — | 0.7 | — | 0.3 |
| Benzyl alcohol | — | 0.5 | — | — |
| Phenoxyethanol | 0.3 | 0.4 | 0.1 | 0.3 |
| Potassium sorbate | 0.2 | — | — | — |
| Sodium benzoate | — | 1.0 | — | 1.0 |
| Allantoin | — | — | 0.5 | 1.0 |
| Sodium lauryl sulfate | — | 0.1 | 0.2 | 0.2 |
| Sodium myristoyl | 1.0 | 1.5 | — | 2.0 |
| sarcosinate | | | | |
| Sodium saccharin | 0.5 | 0.5 | 0.5 | 0.5 |
| Tocopheryl acetate | — | 0.1 | — | 1.0 |
| Zinc chloride | 0.1 | 0.1 | — | 0.5 |
| Carrageenan | 0.1 | 3.0 | — | 0.5 |
| Xanthan gum | — | 0.2 | 0.3 | 0.3 |
| Remainder: | | | | |
| demineralized water | | | | |

Further examples of the composition of oral care compositions according to the invention in the form of toothpastes correspond to the formulations of examples 1-4 given above, but contain the same amount of amorphous calcium phosphate instead of hydroxyapatite.

### Example 2

Examples of the composition of oral care compositions according to the invention in the form of mouth rinses (in % by weight):

| | Example 1 | Example 2 | Example 3 | Example 4 |
|---|---|---|---|---|
| Hydroxyethyl cellulose | — | 0.1 | — | 0.5 |
| Zinc PCA | 0.5 | 0.5 | 0.3 | 0.1 |
| Xylitol | 1.0 | 0.1 | 2.0 | — |
| Dimethyl glycine | 0.01 | 3.5 | 0.5 | 2.5 |
| Sweetener | 0.01 | 0.005 | 0.0001 | 0.1 |
| Glycerol | 1 | 10 | 5 | — |
| Sodium methylcocoyl | 0.5 | 0.2 | — | — |
| taurate | | | | |
| Sodium myristoyl | — | — | 0.3 | 0.1 |
| sarcosinate | | | | |
| Sodium lauryl sulfate | — | — | — | 0.1 |
| Hydroxyapatite | 0.1 | — | 5.0 | — |
| Sorbitol | — | 0.1 | 0.2 | — |
| Flavour | 0.5 | 0.1 | 0.01 | 0.3 |
| Cellulose gum | 1 | 0.2 | 5 | — |
| Sodium benzoate | 0.02 | 0.03 | — | — |
| Phenoxyethanol | — | 0.1 | 0.2 | — |
| Remainder: | | | | |
| demineralized water | | | | |

Further examples of the composition of oral care compositions according to the invention in the form of mouth rinses correspond to the formulations of examples 1-4 given above, but contain the same amount of amorphous calcium phosphate instead of hydroxyapatite.

### Example 3

In an application study, the effect of a toothpaste with dimethylglycine was assessed in comparison with a dimethylglycine-free placebo toothpaste with regard to the occurrence of bleeding gums.

The composition of the toothpaste with dimethylglycine corresponded to the formulation of implementation example 1 from example 1 with 20% hydroxyapatite, the dimethylglycine-free placebo toothpaste contained demineralized water instead of dimethylglycine.

In a 12-week double-blind study, 50 adult subjects aged 40-75 years with bleeding gums were included. The subjects brushed twice daily (morning and evening) for 2 minutes with the toothpastes assigned to them using an electric toothbrush with a round head (25 subjects: verum; 25 subjects control). Supplementary mouth rinses, toothpastes etc. were not allowed to be used during the study period. Only subjects who had at least 10 naturally healthy teeth and were non-smokers were included. Subjects with severe periodontal disease were excluded. The primary examination parameter was the “bleeding on probing index”.

The results at baseline and after 12 weeks of continuous use are shown below:

| | Bleeding on probing index (mean values, in %) | | |
|---|---|---|---|
| | Baseline | After 12 weeks | Improvement |
| Toothpaste with | 18.5 | 9.3 | 9.2 |
| dimethylglycine | | | |
| Toothpaste without | 17.9 | 14.1 | 3.8 |
| dimethylglycine | | | |
| (placebo) | | | |

In an application study, the use of a toothpaste with dimethylglycine led to a significant reduction in bleeding gums compared to a dimethylglycine-free placebo toothpaste. A subject questionnaire issued after 12 weeks showed that the toothpaste with dimethylglycine was widely accepted in terms of taste, tolerability and mouthfeel after brushing.

### Example 4

As part of an in vitro study, the effect of sodium dimethylglycinate was investigated in a cell culture model with human keratin-forming keratinocyte cells. For this purpose, HaCaT cells were cultured for 1, 3, 5 and 7 days in DMEM medium (including fetal calf serum and an antibiotic-antimycotic mix) and the viability, proliferation and migration of the cells were determined using suitable measurement methods and the expression of growth factors relevant for cell growth was determined.

### Detection of Viability:

For this measurement, a so-called MTT assay was used to determine cellular metabolic activity as an indicator of cell viability and cytotoxicity. This colorimetric assay is based on the reduction of a yellow tetrazolium salt (3-(4,5-dimethylthiazol-2-yl)-2,5-diphenyltetrazolium bromide or MTT) to purple formazan crystals by metabolically active cells.

Human epidermal keratinocyte cells (HaCaT) were seeded in a 96-well plate at a cell density of 5,000 cells/well and cultured in medium (DMEM with 10% FBS, 1% penicillin-streptomycin, 0.5% fungizone). On the next day and after a further 24 h, 48 h and 72 h of cultivation at 37° C. and 5 vol. % CO₂, the cell culture medium without (control) or with the respective concentrations of sodium dimethylglycinate (DMG) already contained was changed. The measurement was performed analogously to previously published studies in B. I. Tóth, N. Dobrosi, A. Dajnoki, G. Czifra, A. Oláh, A. G. Szöllösi, I. Juhász, K. Sugawara, R. Paus, T. Biró, J Invest Dermatol 2011, 131, 1095-1104.

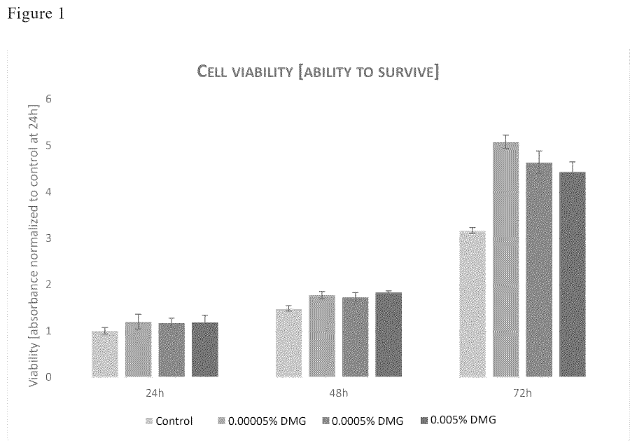

FIG. 1 shows the cell viability after 24 h, 48 h and 72 h of cultivation and a clear increase after 48 h and 72 h due to the addition of DMG is observed. Cell viability was determined using the MTT assay. The absorption was measured in quadruplicate and normalized to the control at 24 h. The mean value with standard deviation is indicated.

### Proof of Proliferation:

A so-called CyQUANT assay was used to measure proliferation. In this fluorescence-based assay, the fluorescent dye used binds to DNA (deoxyribonucleic acid), whereby the content of cellular DNA is a direct measure of the number of cells within a sample.

Human epidermal keratinocyte cells (HaCaT) were seeded in a 96-well plate at a cell density of 5,000 cells/well and cultured in medium (DMEM with 10% FBS, 1% penicillin-streptomycin, 0.5% fungizone). On the next day and after a further 24 h, 48 h and 72 h of cultivation at 37° C. and 5 vol. % CO₂, the cell culture medium without (control) or with the respective concentrations of active substance already contained was changed. The measurement was performed analogously to previously published studies in A. Oláh, B. I. Tóth, I. Borbiró, K. Sugawara, A. G. Szöllösi, G. Czifra, B. Pál, L. Ambrus, J. Kloepper, E. Camera, The Journal of clinical investigation 2014, 124, 3713-3724.

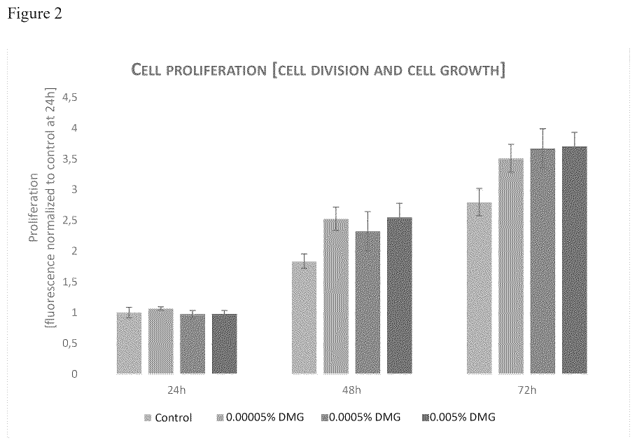

FIG. 2 shows the cell proliferation after 24 h, 48 h and 72 h of cultivation and a clear increase after 48 h and 72 h due to the addition of DMG is observed. Cell proliferation was determined using the CyQUANT assay. The fluorescence was measured in triplicate and normalized to the control at 24 h. The mean value with standard deviation is indicated.

### Proof of Migration:

A so-called wound healing assay was used to measure migration, based on previously published studies in T. Kawabata, T. Otsuka, K. Fujita, G. Sakai, R. Matsushima-Nishiwaki, O. Kozawa, H. Tokuda, International journal of molecular medicine 2018, 42, 3149-3156. The principle is based on measuring the migration of cells to a not yet colonized cultivation surface over time. For this purpose, 20,000 cells each were seeded into two adjacent wells or cavities separated by a silicone insert (with standardized width) and cultivated in medium (DMEM with 5% FBS, 1% penicillin-streptomycin, 0.5% fungizone) for 48 h at 37° C. and 5 vol % CO₂. The plastic insert was then removed (“creation of the wound”) and the migration of the cells was documented by determining the cultivation surface not colonized with cells over time using images. In parallel, the cell culture medium without (control) or with the respective concentrations of active substance already contained was changed directly after removal of the plastic insert (0 h) and after a further 24 h. The evaluation of the images and determination of the cultivation surface not colonized with cells was carried out using specific software (ImageJ).

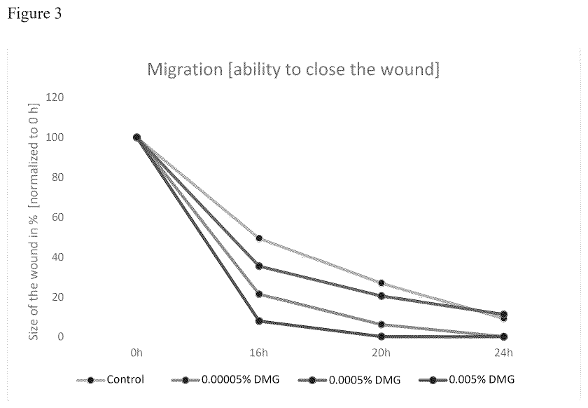

FIG. 3 shows the migration over time and using the different DMG concentrations. Migration or wound closure is each significantly faster with DMG compared to the control without DMG. The wound was created at 0 h and images were taken after 16 h, 20 h and 24 h of cultivation. Based on the images, the cultivation surface that was not yet colonized with cells was determined using the ImageJ software. The measured values are normalized to the time point 0 h (maximum size of the wound) and given in %.

### Detection of VEGF Gene Expression:

VEGF (vascular endothelial growth factor) promotes the growth and formation of new blood and lymph vessels. Gene expression was measured using a standard method known as quantitative real-time PCR (qRT-PCR).

Human epidermal keratinocyte cells (HaCaT) were seeded in a 6-well plate at a cell density of 140,000 cells/well and cultured in medium (DMEM with 10% FBS, 1% penicillin-streptomycin, 0.5% fungizone) at 37° C. and 5 vol % CO₂. On the next day, the cell culture medium without (control) or with the respective concentrations of active substance already contained was changed and the cells were harvested after 24 hours. Gene expression was measured using qRT-PCR based on previously published studies in B. V. Diaz, M.-C. Lenoir, A. Ladoux, C. Frelin, M. Demarchez, S. Michel, Journal of Biological Chemistry 2000, 275, 642-650.

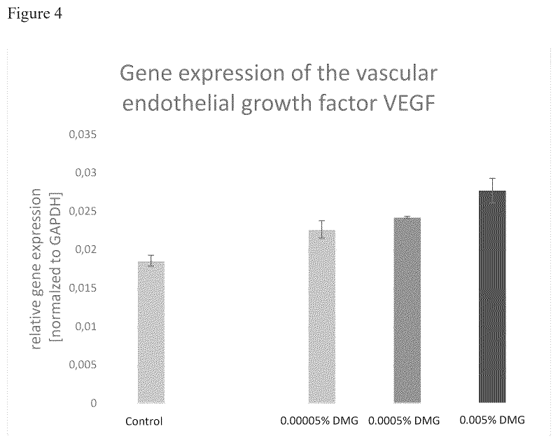

FIG. 4 shows the gene expression of VEGF after 24 h of cultivation and a significant increase in gene expression due to the addition of DMG is observed. The relative gene expression of VEGF determined using qRT-PCR (triplicate determination, normalized to the respective gene expression of a constitutively expressed gene; GAPDH—glyceraldehyde-3-phosphate dehydrogenase) is shown. The mean value with standard deviation is indicated.

Surprisingly, it was shown that DMG had a positive effect on the growth-relevant parameters of HaCaT cells and that the expression of the growth factor VEGF was significantly increased compared to the treatment of HaCaT cells without DMG.

### Detection of the Gene Expression of Interleukin 6:

Interleukin-6 (IL-6) is a peptide hormone of the immune system from the group of pro-inflammatory interleukins and plays a key role in the non-specific, innate immune response. Gene expression was measured using a standard method known as quantitative real-time PCR (qRT-PCR).

Human epidermal keratinocyte cells (HaCaT) were seeded in a 6-well plate at a cell density of 140,000 cells/well and cultured in medium (DMEM with 10% FBS, 1% penicillin-streptomycin, 0.5% fungizone) at 37° C. and 5 vol % CO₂. On the next day, cytokine treatment (25 ng/ml tumor necrosis factor-alpha (TNFα) and 25 ng/ml interferon-gamma (IFNγ)) was used to induce cell inflammation. After 1, treated with the different DMG drug concentrations (inflammation induction+DMG 0.005%−0.00005%) or without DMG (positive control of inflammation induction). In parallel, cells were treated without any treatment (control) or only with the different DMG drug concentrations (DMG 0.005%−0.00005%) and the cells were harvested after 24 h for gene expression analysis. Gene expression was measured using qRT-PCR based on previously published studies in B. V. Diaz, M.-C. Lenoir, A. Ladoux, C. Frelin, M. Démarchez, S. Michel, Journal of Biological Chemistry 2000, 275, 642-650.

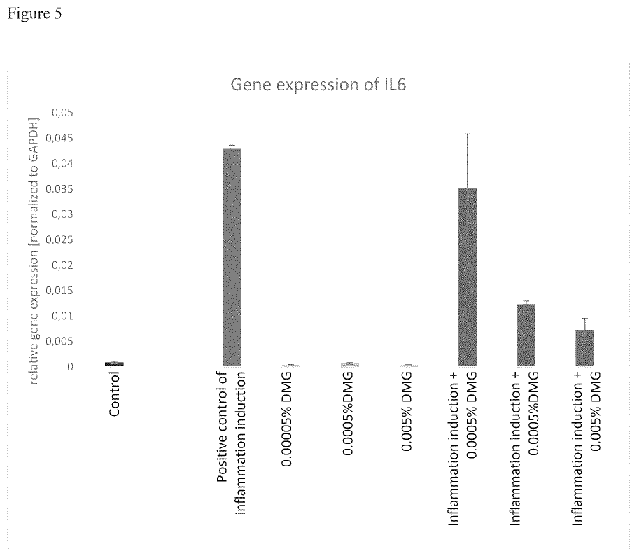

FIG. 5 shows the gene expression of IL-6 after 24 h of cultivation. The relative gene expression of IL-6 determined by qRT-PCR (triplicate determination, normalized to the respective gene expression of a constitutively expressed gene; GAPDH—glyceraldehyde-3-phosphate dehydrogenase) is shown. The mean value with standard deviation is indicated.

Surprisingly, the expression of interleukin-6 induced by cytokine treatment was dose-dependently reduced by the addition of DMG compared to the positive control of inflammation induction.

1. Oral care composition, characterized in that the oral care composition contains dimethylglycine and/or a salt thereof.

2. Oral care composition according to claim 1, characterized in that the oral care composition contains dimethylglycine and/or a salt thereof in an amount of from 0.00001 to 10.0% by weight, preferably from 0.0001 to 2.0% by weight and particularly preferably from 0.0005 to 1% by weight, in each case based on the total weight of the oral care composition.

3. Oral care composition according to one of the preceding claims, characterized in that the oral care composition further contains a calcium phosphate compound.

4. Oral care composition according to claim 3, characterized in that the calcium phosphate compound is present in particulate form and/or in that the calcium phosphate compound is selected from the group consisting of monocalcium phosphate monohydrate (MCPM), monocalcium phosphate anhydrate (MCPA), dicalcium phosphate dihydrate (DCPD, brushite), dicalcium phosphate anhydrate (DCPA, monetite), octacalcium phosphate (OCP), α-tricalcium phosphate (α-TCP), β-tricalcium phosphate (β-TCP), amorphous calcium phosphate (ACP), calcium-deficient hydroxyapatite (CDHA), hydroxyapatite (HA or HAP), tetracalcium phosphate (TTCP) and calcium pyrophosphate and/or wherein the calcium phosphate compound is preferably hydroxyapatite.

5. Oral care composition according to claim 3 or claim 4, characterized in that the oral care composition comprises the calcium phosphate compound in an amount of 0.01 to 40.0% by weight, preferably 0.1 to 30.0% by weight, more preferably 0.5 to 20.0% by weight, based on the total weight of the oral care composition.

6. Oral care composition according to one of the preceding claims, characterized in that the oral care composition further comprises a surfactant, wherein the surfactant is preferably selected from the group consisting of betaines, glycinates, sarcosinates, sulfates and taurates.

7. Oral care composition according to one of the preceding claims, characterized in that the oral care composition further comprises antibacterial substances and/or cleaning agents.

8. Use of the oral care composition according to any one of claims 1 to 7 for the care of the oral and pharyngeal soft tissues.

9. Use of the oral care composition according to any one of claims 1 to 7 for the prevention and/or therapy of oral diseases selected from gingivitis, periodontitis, mucositis, peri-implantitis, peri-implant mucositis, aphthae, fungal thrush, oral fistulae, receding gums, bad breath, ulcers, saliva deficiency.

10. Use of the oral care composition according to any one of claims 1 to 7 for improving the oral soft tissues.

11. Use of the oral care composition according to any one of claims 1 to 7 for the prevention and/or therapy of a bacterial and/or viral infection and/or mycoses.

12. Oral care composition according to any one of claims 1 to 7, characterized in that the oral care composition is a toothpaste, tablet, chewing gum, oral gel, gel for gingival pockets, mouth rinse or mouthwash.
